(19) United States
(12) Patent Application Publication
(10) Pub. No.: US 2004/0064157 A1
(43) Pub. Date: Apr. 1, 2004

(54) Method and apparatus for maintaining energy storage in an electrical storage device

(76) Inventor: John D. Norton, New Brighton, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/261,066

(22) Filed: Sep. 30, 2002

(51) Int. Cl. A61N 1/39

(52) U.S. Cl. 607/7

(57) ABSTRACT

A method and an apparatus for performing maintaining a charge in an electrical energy storing device, such as a capacitor. A set-point voltage for the electrical energy storage device is used during an initial device charging process to charge the storage device to the set-point voltage. A charge maintenance process is also performed that offsets charge loss due to leakage currents, polarization currents and the like. The charge maintenance process involves providing a charge maintenance current to the storage device until the storage device is therapeutically discharged (e.g., during cardiac defibrillation therapy delivery).

### CROSS REFERNCE TO RELATED APPLICATION

This non-provisional U.S. patent application is related to a non-provisional U.S. patent application Ser. No. 10/XXX,XXX filed on even date hereof, invented by Norton and Keimel, and entitled “Apparatus and Method for Optimizing Capacitor Charge in a Medical Device,” the contents of which are hereby incorporated by reference herein.

### BACKGROUND OF THE INVENTION

### FIELD OF THE INVENTION

This invention relates generally to methods and apparatus for precisely controlling the amount of energy delivered from one or more capacitors; in particular, methods and medical apparatuses for precisely delivering anti-arrhythmia cardiac therapies from one or more capacitors.

### DESCRIPTION OF THE RELATED ART

The technology explosion in the implantable medical devices industry has resulted in many new and innovative devices and methods for analyzing and improving the health of a patient. The class of implantable medical devices now includes pacemakers, implantable cardioverter-defibrillators, neural stimulators, and drug administering devices, among others. Today's state-of-the-art implantable medical devices are vastly more sophisticated and complex than early ones, capable of performing significantly more complex tasks. The therapeutic benefits of such devices have been well proven.

There are many implementations of implantable medical devices that provide data acquisition of important physiological data from a human body. Many implantable medical devices are used for cardiac monitoring and therapy. Often these devices comprise sensors that are placed in blood vessels and/or chambers of the heart. Often these devices are operatively coupled with implantable monitors and therapy delivery devices. For example, such cardiac systems include implantable heart monitors and therapy delivery devices, such as pace makers, cardioverter, defibrillators, heart pumps, cardiomyostimulators, ischemia treatment devices, drug delivery devices, and other heart therapy devices. Most of these cardiac systems include electrodes for sensing and gain amplifiers for recording electrical activity and/or driving electrical pacing signals based on electrodes disposed subcutaneously e.g., on an implantable the device housing, on a medical electrical lead (known as EGM) or using surface electrodes adhered to the skin of a patient (ECG).

As the functional sophistication and complexity of implantable medical device systems have increased over the years, it has become increasingly useful to include efficient and varied forms of energy delivery systems in the medical device. For example, today's implantable medical devices may deliver a number of types of cardiac therapy in a patient. Furthermore, cardiac therapy systems now provide for more rapid/frequent delivery of therapy. Conventional energy delivery systems in an implantable medical device system can be inefficient when the timing of therapy delivery varies. An increase in the time period between charging an electrical energy storage device, such as a capacitor, and therapy delivery may cause inefficiencies in maintaining optimum charge due primarily to leakage current(s). This can cause degradation in the intensity of the therapy delivered. As a result, a decrease in the magnitude of the delivered energy may occur. One way to compensate for these effects is to utilize larger components in the devices than are actually required, thereby increasing the overall volume of the implantable medical devices.

An implantable cardioverter-defibrillator (ICD) typically employs one or more high-voltage capacitors to deliver suitably timed defibrillation therapies. Typically, each capacitor is charged to a voltage set point, and then the charge process is generally terminated. Often, there is some time lag between the end of a capacitor charge process and the moment defibrillation therapy is delivered. In some cases, as many as several seconds may pass between the end of the charge process and the beginning of the therapy delivery. During this time lag, the voltage (charge) held by the capacitor may decrease as described above.

As noted, capacitor voltage may decrease due to current leakages. The leakage current may result from a true electronic/ionic leakage across the dielectric, resulting in loss of stored charge and a decrease in the capacitor voltage. The leakage current may also be due to a continuing creation and alignment of dipoles within the dielectric materials of the capacitor. It is believed that the processes that lead to dielectric polarization may have a range of time constants, including some which are actually longer than the time required to charge the capacitors. Therefore, polarization of the dielectric continues under an applied field during, and after completion of, the charging of the capacitor. This may decrease the field within the dielectric, therefore decreasing the applied voltage. When the applied voltage in the capacitor is decreased, the therapy delivered from the discharging of the capacitor can be compromised. This phenomenon is known as “dielectric polarization” or “dielectric absorption.”

The present invention is directed to overcoming, or at least reducing the effects of, one or more of the problems set forth above.

### SUMMARY OF THE INVENTION

The present invention provides an electrical circuit and method for charging capacitors which result in increased (i) energy stored in the capacitors, (ii) energy delivered to cardiac tissue of a patient and (iii) the leading edge voltage of the delivered energy. Incidental to these aspects of the present invention, but of utmost importance with respect to implantable medical devices that provide defibrillation therapy, the present invention results in relatively compact devices which deliver more defibrillation energy than comparable albeit prior art devices. This important aspect of the invention vis-à-vis implantable medical devices can be understood in light of the fact that generally speaking the amount of energy stored (and delivered) by a capacitor varies with the mathematical square of the voltage multiplied by the capacitance of the capacitor (i.e., 1/2CV²). Thus, it can be appreciated that if the actual voltage of a capacitor at the time of discharge is precisely held at the designed (i.e., desired) charge set point, circuit designers will not have to anticipate any loss of voltage (e.g., due to polarization current, true leakage current, and the like) between the initial charge and the moment the capacitor is discharged. With respect to implantable medical devices this translates into a reduced overall capacitor volume as compared to prior art approaches. While the present invention has particular utility for such implantable devices, external pacing and/or defibrillation devices (e.g., automatic external defibrillators) may advantageously employ the teaching of the invention and are intended to be covered hereby. In addition, while perhaps not the primary beneficiaries of the teaching herein, the invention is also intended to cover very low voltage capacitors, such as are routinely employed to provide low voltage cardiac pacing therapy. In general, any capacitor that loses charge after being fully charged may benefit from the teaching hereof; in particular, any electrolytic capacitor or solid state capacitor and the like may be improved according to the present invention. With respect to the improved utility for such capacitors, any capacitor application wherein precision in the amount of energy actually delivered by a capacitor is desired (or required) will benefit from this invention.

In one aspect of the present invention, a method is provided for maintaining energy stored in an energy storage/discharge device. A set-point for charging the electrical energy storage device is determined or preset. An initial full-charging process to charge the electrical energy storage device to the set-point is performed. A charge maintenance process is then performed following the full-charging process, the charge maintenance process comprising providing a charge maintenance electrical current to compensate for the loss of charge (i.e., the actual charge falling below the set-point). Ideally the charge maintenance process is a dynamic feedback so that the magnitude of the maintenance charge precisely offsets all sources of the loss of charge. However, in a practical and preferred embodiment, a voltage comparator monitors the actual charge present and the charge maintenance current is supplied when the monitored charge decreases below a lower threshold (e.g., a floor voltage of very nearly the same magnitude of the set-point voltage). That is, the charge maintenance current cycles on and off (e.g., depending on the timing, set-point, floor voltage and circuitry the charge maintenance current may resemble a periodic or aperiodic sawtooth pattern, a sinusoidal pattern and the like). In this way, the present invention provides an indirect albeit practical surrogate for actual charge loss due to leakage currents, polarization currents, etc. In an ICD, for example, the charge maintenance current may result from current delivered to the primary winding of a high voltage transformer such that when the current ceases a secondary current forms in the secondary winding and is then delivered to the capacitor. Those of skill in the art will recognize myriad ways of implementing this aspect of the present invention and each way is intended to be covered hereby.

In another aspect of the present invention, an apparatus is provided for maintaining energy stored in an energy storage/discharge device. The apparatus of the present invention comprises: a processor; a control logic operatively coupled to the processor, the control logic to generate at least one control signal in response to a command from the processor; a capacitor unit operatively coupled to the control logic, the capacitor unit to acquire a charge and release the charge in response to the control signal; and a charge maintenance unit operatively coupled to the capacitor unit, the charge maintenance unit to provide a charge maintenance current to the capacitor unit.

In yet another aspect of the present invention, a computer readable program storage device encoded with instructions is provided for maintaining energy stored in an energy storage/discharge device. The computer readable program storage device encoded with instructions: determines a set-point for charging a capacitor; performs a charging process to charge the capacitor in response to the determining of the set-point; and performs a charge maintenance process in response to the charging process. The charge maintenance process comprises: monitoring a voltage level of the capacitor; determining whether the voltage level is below a predetermined threshold; providing a charge maintenance current to the capacitor in response to a determination that the voltage level is below a predetermined threshold; and terminating the charge maintenance current in response to a determination that the voltage level is not below a predetermined threshold or in response to a discharge-capacitor instruction.

More generally, the circuit and techniques of the present invention operate so that a first charging electrical current is applied to a capacitor and, upon reaching a predetermined set-point voltage for a capacitor, a second, lesser charging electrical current (i.e., the maintenance electrical current referred to above) flows into the capacitor. The maintenance electrical current preferably is of magnitude sufficient to maintain the charge at or very nearly equal to the set-point and in balance with the leakage current of the capacitor or oscillates between a floor voltage and the set-point (or ceiling voltage).

### BRIEF DESCRIPTION OF THE DRAWINGS

The various aspects of the present invention may be understood by reference to the following description taken in conjunction with the accompanying drawings, which are not drawn to scale and in which like reference numerals identify like elements.

FIG. 1 is a simplified diagram of an implementation of an implantable medical device, in accordance with one illustrative embodiment of the present invention.

FIG. 2 illustrates a simplified diagram representation of an implantable medical device system in accordance with one illustrative embodiment of the present invention.

FIG. 3 depicts an exploded view of an exemplary implantable medical device adapted to utilize the present invention.

FIG. 4 illustrates an interaction between a sensor and the implantable medical device of FIG. 2, in accordance with one illustrative embodiment of the present invention.

FIG. 5 is a flowchart depicting a method of practicing the present invention.

FIG. 6 illustrates a more detailed flowchart depiction of a method of charging-up of a capacitor for eventual discharge in an implantable medical device, as indicated in FIG. 5, in accordance with one illustrative embodiment of the present invention.

FIG. 7 illustrates a more detailed flowchart depiction of an alternative method of performing a charge-maintenance process, as indicated in FIG. 5, in accordance with one illustrative embodiment of the present invention.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Illustrative embodiments of the present invention are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

Many times, due to current leakage, dielectric polarization, and other electrical phenomenon, a portion of an initial full charge held by a capacitor is inadvertently lost. In the context of electrical cardiac therapy delivery, this can compromise the energy of the therapy delivered to a patient, thereby compromising the effectiveness of the therapy and/or resulting in oversized capacitors and therefore, increased device volume. Embodiments of the present invention provide methods and apparatus for applying a charge maintenance circuit capable of providing post-charge energy to a capacitor for maintaining the intended charge level in the capacitor prior to discharge of the capacitor. The present invention provides particular benefits when used in conjunction with so-called standard capacitors that have appreciable leakage electrical current (in addition to polarization current), although so-called low leakage capacitors will benefit from application of the teaching of the present invention.

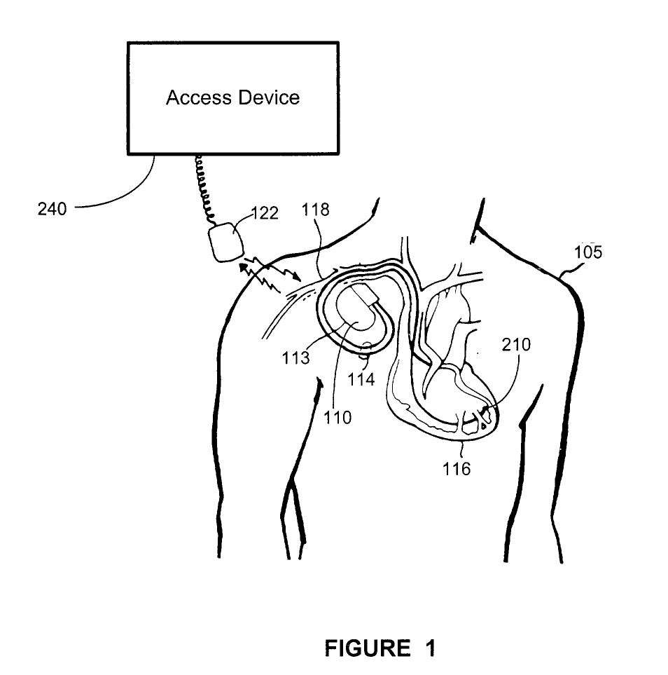

FIG. 1 illustrates one embodiment of implementing an implantable medical device into a human body. A sensor/therapy delivery device 210 (e.g., ring, tip and/or coil electrode coupled to lead 114) is guided to a location on, in or proximate one or more chamber of a heart 120 of a patient 105. The device 210 is used to sense physiologic data and/or provide electrical therapy to tissue of the heart 120. As is known in the art, an implantable medical device 110 collects and processes a plurality of data acquired from the human body. The implantable medical device 110 may be a pacemaker, neurological stimulator, a muscle stimulator, a drug pump or a combination of the foregoing that preferably has a cardiac defibrillation capability; however, in the preferred embodiment the device 110 is an implantable cardioverter-defibrillator (ICD). The data acquired by the implantable medical device 110 can be monitored by an external system, such as the access device 240 comprising a programming head 122, which remotely communicates with the implantable medical device 110 as is known in the art. The programming head 122 is utilized in accordance with medical device programming systems also known to those skilled in the art, for facilitating two-way communication between the implantable medical device 110 and the access device 210.

In one embodiment, a plurality of access devices 240 can be employed to collect a plurality of data processed by the implantable medical device 110 in accordance with embodiments of the present invention. The implantable medical device 110 is housed within a hermetically sealed, biologically inert outer canister or housing 113, which may itself be conductive so as to serve as an electrode in the implantable medical device's pacing/sensing and/or defibrillation circuit. One or more implantable medical device sensors/leads, collectively identified with reference numeral 114 in FIG. 1 are electrically coupled to the implantable medical device 110 and extend to tissue of a patient's heart 116 (e.g., to peridcardial tissue, to myocardial tissue, to endocardial tissue via a vein 118 such as the superior vena cava or to epicardial tissue). Disposed generally near a distal end of the leads 114 are one or more exposed conductive electrodes 210 for receiving electrical cardiac signals and/or delivering electrical pacing stimuli to the heart 116. The leads 114 oftentimes are implanted with their distal end situated in either (or both) the atrium or ventricle(s) of the heart 116. In an alternative embodiment, the sensors 210, or the leads 114 associated with the sensors 210, may be situated in a blood vessel on the heart 116, such as a vein 118. While FIG. 1 depicts an implantable medical device 110, device 110 may be an external medical device (e.g., an automatic external defibrillator and the like). Also, as is known in the art, the canister, or can, portion of the implantable medical device can serve as an active electrode and thus form a part of a defibrillation therapy circuit in cooperation with one or more of the electrodes.

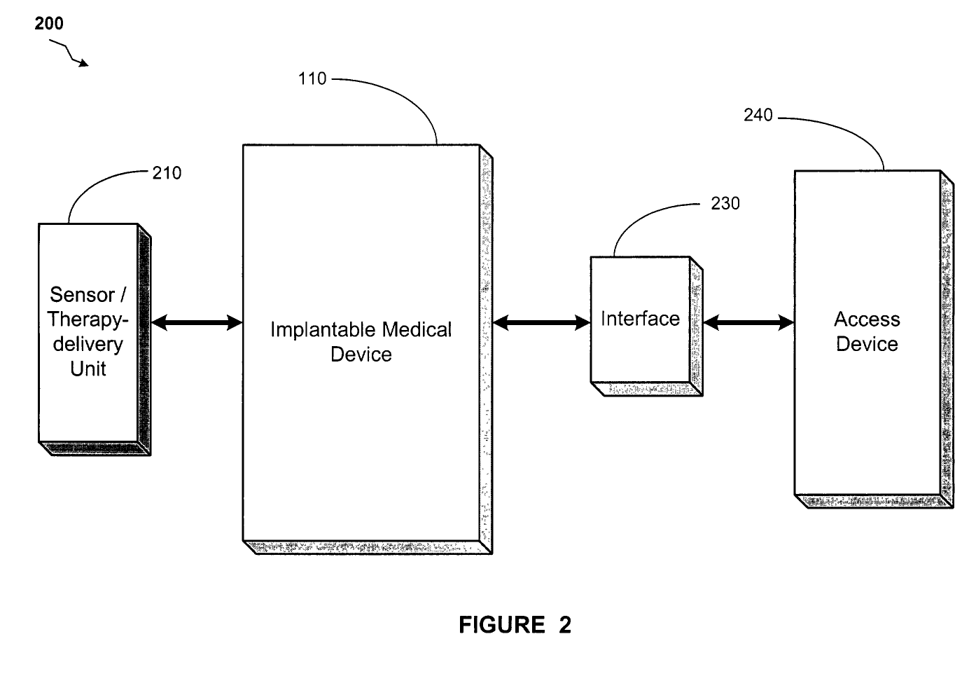

Turning now to FIG. 2, a system 200, in accordance with one embodiment of the present invention, is illustrated. The system 200 comprises a sensor/therapy delivery device 210, an implantable medical device 110, an access device 240, and an interface 230 that provides a communication link between the implantable medical device 110 and the access device 240. Embodiments of the present invention provide a plurality of physiological data from the sensor 210, which are then processed and stored in the implantable medical device 110. Based upon physiologic data and other factors, the implantable medical device 110 may deliver a therapy to a portion of the patient's body, via the sensor/therapy delivery device 210. The access device 240 can then be used to monitor and analyze the data from the implantable medical device 110 via the interface 230 and view results from delivered therapy. The access device 240 can be used to monitor the efficiency of the therapy delivered by the implantable medical device 110. The access device 240 can be used to detect an arrhythmia such as an atrial or a ventricular fibrillation, based upon data sensed by the implantable medical device 110, to determine whether a defibrillation therapy needs to be delivered. The magnitude of such a defibrillation therapy may be programmed to vary for successive delivery of therapy or may vary depending on the type of arrthymia detected. For example, a cardioversion therapy may require several joules of energy while an atrial defibrillation therapy may require tens of joules and a ventricular defibrillation may require 20 to 30 joules of energy to effectively terminate such a (potentially deadly) ventricular arrthymia. A look up table or the like may be used or programmable values may be used to determine the amount of energy delivered for a particular occurrence of such arrhythmias. In addition, the type of waveform used to apply the energy may be selectable, in advance or pre-programmed so that alternating types of waveforms are applied in an attempt to terminate an arrthymia. Two basic types of waveforms are the monophasic and the biphasic, as is well known in the art.

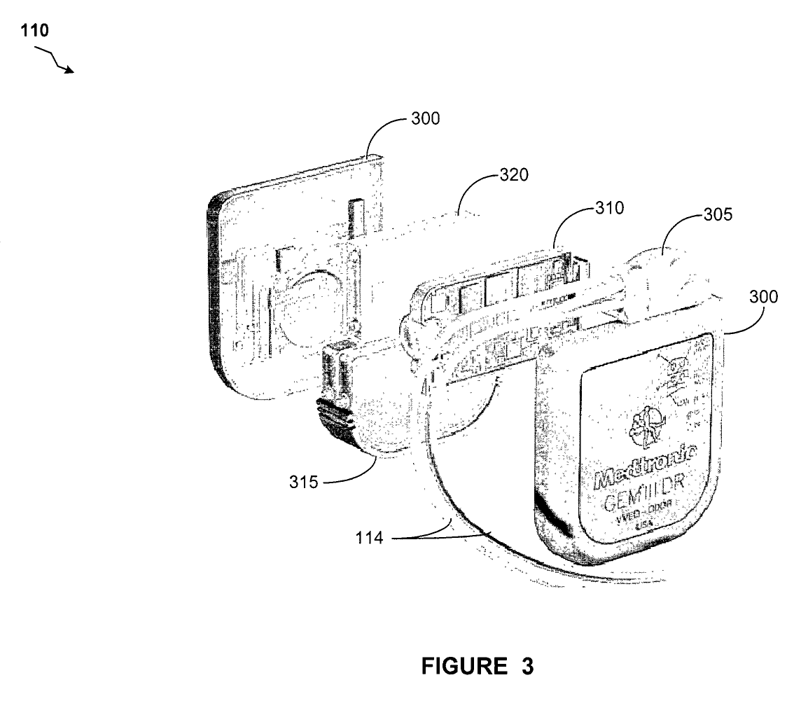

Turning now to FIG. 3, a stylized three-dimensional representation with several sub-assemblies depicted in an exploded view of the implantable medical device 110 is illustrated. In one embodiment, a casing 300 may include a variety of elements including, but not limited to, a connector 305, a processor unit 310, an electrical energy storage device such as a capacitor package 315 having a pair of capacitors coupled in series, and a primary power source 320, such as a battery. The processor unit 310 is preferably coupled to transformer circuitry for charging the capacitor(s) of the package 315 to a predetermined voltage and in general operates the device according to programmable features such as arrthymia detection, therapy delivery, sensing/pacing to maintain stable cardiac rhythm, data logging (e.g., EGM tracing, heart rate, ventilation, etc.), error tracking, telemetry handling, and the like. The elements in the casing 300 may be positioned in any of a variety of locations. The capacitor package 315 and the battery 320 may be electrically coupled to the processor unit 310. The leads 114 may be interfaced with the implantable medical device 110 through the connector 305 and may electrically connect portions of the patient 112 such as the heart 116 to the implantable medical device 110.

The processor unit 310 typically employs sense amplifiers (not depicted) that detect and/or register cardiac signals. In one embodiment, the processor unit 310 uses the sensed cardiac signals to detect when a cardiac event, such as normal sinus rhythm, bradycardia, tachycardia, flutter, or fibrillation, occurs. In response to such a cardiac event or other conditions, the processor unit 310 administers one or a plurality of therapies. Examples of such a therapy include cardioversion therapy and defibrillation therapy. Another example is providing pacing therapy to one or more chambers of the heart 116. The implantable medical devices 110 may deliver the therapy by releasing energy stored in the capacitor package 315 (or smaller dedicated capacitors for pacing—not depicted) and directing the energy through the connector 305 and onto the leads 114, to the heart 116. The capacitor package 315 may comprise one or more capacitors (two are depicted) that may initially store a desired charge equal to a set-point for the capacitor (given the nature of the desired therapy), such that when the charge is released, it can terminate (or convert) a potentially harmful cardiac arrthymia, such as ventricular fibrillation.

The battery 320 provides energy that is used to power the processor unit 310 and to recharge the capacitor package 315, as required. In some cases, due to one or more electrical phenomenon, such as charge loss due to polarization and leakage currents from the capacitor package 315, the capacitor(s) may not retain the desired charge level (i.e., the set-point voltage) for more than a brief instant of time. The present invention reduces the effect(s) of these factors that compromise the energy levels of the initial full charge held by the capacitor package 315 while at the same time reducing the volume of the capacitor package 315.

In the prior art (and as briefly noted above), the capacitor package 315 was essentially oversized to accommodate the effects of leakage and polarization currents. The system provided by embodiments of the present invention may be implemented into a variety of relatively compact implantable medical devices 110 like ICDs, heart pacemakers and drug delivery devices, as well as into non-medical devices that are sensitive to the amount of energy delivered from an electric device such as a capacitor.

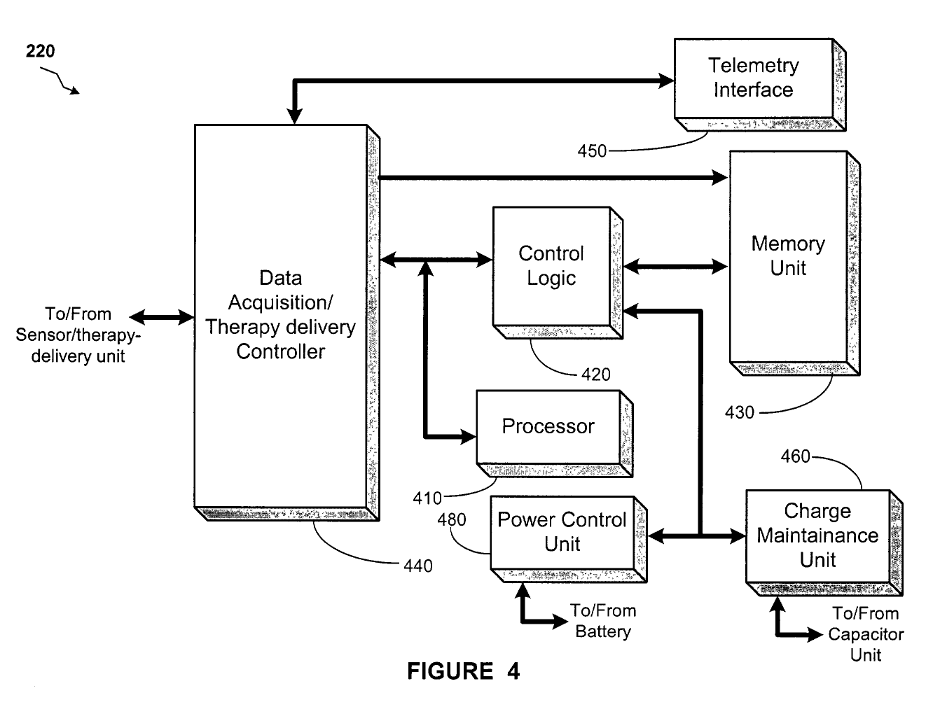

Turning now to FIG. 4, a more detailed block diagram depiction of one embodiment of the implantable medical device 110 is illustrated. In one embodiment, the block diagram shown in FIG. 4 provides a functional illustration of the processor unit 310 of FIG. 3. The implantable medical device 110 comprises a processor 410, a control logic 420, a memory unit 430, a data acquisition controller 440, a telemetry interface 450, a charge maintenance unit 460, and a power control unit 480. In one embodiment, a plurality of the blocks illustrated in FIG. 4 may be integrated as a single unit.

The processor 410 controls the operation of the implantable medical device 110. The processor 410 utilizes the control logic 420 to perform a plurality of operations, including memory access and storage operations. The processor 410 communicates with the control logic 420 and the data acquisition controller 440 via a bus line 425. The control logic 420 sends control signals to the memory unit 430 for controlling and installing memory 430, and to the data acquisition controller 440, which controls the acquisition of physiological data, delivery of therapy, and drives output signals to the telemetry interface 450.

The telemetry interface 450 can facilitate access to physiological data previously acquired by the data acquisition controller 440. Therefore, a physician can view physiological data by accessing the data acquisition controller 440, via the telemetry interface 450. The data acquisition controller 440 can prompt the implantable medical device to acquire physiological data and/or deliver a cardiac therapy.

The implantable medical device 110 utilizes the power control unit 480, which may be coupled to the power source 320, and a charge maintenance unit 460, which may be electrically coupled to the capacitor unit 315, to deliver a therapy (e.g., a cardiac therapy) to the patient 112. In one embodiment, the processor 410 controls the operations of the power control unit 480 and the charge maintenance unit 460 via the control logic 420. The processor 410 monitors physiological data received by the data acquisition controller 440 and reacts accordingly. For example, when the processor 410 detects an arrhythmia (e.g., tachycardia, fibrillation, etc.), the processor 410 may invoke a therapeutic response precisely timed to coincide with the cardiac cycle. The processor 410 then prompts the control logic 420 to invoke the power control unit 480. In response, the power control unit 480 may cause the power source 320 to deliver an appropriate amount of charge or energy, which may be pre-programmed, to the capacitor unit 315 (e.g., via a high voltage, step-up transformer unit coupled to the power source 320).

A suitable, preferred high-rate battery power source 320 (including the transformer) can rapidly charge the capacitor unit 315 (e.g., typically within several seconds by applying on the order of ten amps of current). The charge applied to capacitor unit 315 may depend upon a predetermined time period during which a charge is provided by the power source 320, but typically is constrained to a pre-selected set-point voltage. The period of time for the capacitor unit 315 to charge largely depends on the chosen voltage set-point (and the time constant associated with the capacitor unit 315). In order to obtain and/or maintain a desired charge, the control logic 420 preferably invokes the charge maintenance unit 460 promptly following completion of the initial full charge of capacitor 315. As described above, the initial full charge in the capacitor unit 315 may immediately be reduced due to losses caused by leakage current and/or polarization current, among other factors.

In one form of the invention, the charge maintenance unit 460 begins providing the charge maintenance current immediately after the capacitor unit 315 is charged to a desired voltage set-point. The charging current may either comprise an electrical current of magnitude sufficient to offset the leakage and polarization currents or may be temporarily applied so that the capacitor retains a charge between a floor voltage and a ceiling voltage. In the latter case, the ceiling voltage may be equal to the set-point with the floor voltage set to a value very close to the set-point value. In other words, the charging current is provided at a magnitude sufficient to overcome the dielectric polarization and the leakage current associated with the capacitor unit 315.

In another form of the invention, the charge maintenance unit 460 is capable of detecting the amount of charge needed, the approximate amount of leakage current, and losses due to dielectric polarization (i.e., polarization currents). The charge maintenance unit 460 may then determine the magnitude of charging current required to maintain the predetermined charge.

In yet another form of the invention, the charge maintenance unit 460 will resume charging the capacitor unit 315 at periodic intervals after the desired voltage set-point is reached. Each charging episode continues until the desired voltage set-point is re-attained.

In one embodiment, the system 200 cycles the power supply directly from the power source 320 (as opposed to the preferred route including the high voltage circuitry, step-up transformer and the like) on and off to maintain the voltage in the capacitor unit 315 for either a pre-determined period of time or such time as the capacitor is discharged during therapy delivery. In one form of this embodiment, the charge maintenance unit 460 causes the current to the capacitor unit 315 to start and stop, depending on the charge level of the capacitor unit 315 relative to a set-point, in order to maintain a predetermined charge level. The capacitor charge is thus maintained at or very near the set-point for a time period, which in one embodiment relates to a time period that ends when the capacitor unit 315 is discharged to deliver a therapy, such as a cardiac defibrillation therapy. In the context of cardiac defibrillation therapy, such a period of time may approach 30 seconds or more.

The offset of the charge loss in the capacitor unit 315 results in a more precise amount of energy delivered by the capacitor unit 315 to the patient 112. Accordingly, the leading edge voltage of the defibrillation waveform delivered to the patient 112 is more precisely controlled (at a predetermined value). Furthermore, the ability to maintain an adequate charge in the capacitor unit 315 provides for using a capacitor package 315 of smaller volumetric size relative to prior art packages. This allows the overall size of the implantable medical device 110 to be reduced. Additionally, utilization of charge maintenance unit 460 provides for more efficient use of the power in the power source 320 and in the capacitor unit 315, such that the battery life of the power source 320 may be extended as compared to prior art devices.

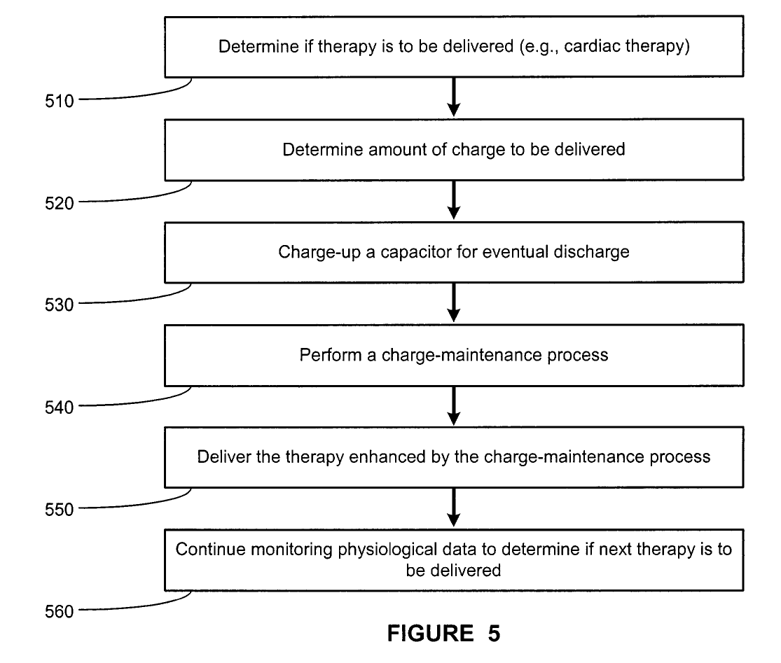

Turning now to FIG. 5, a flowchart depiction of the method in accordance with one embodiment of the present invention is illustrated. In one embodiment, the implantable medical device 110 determines if a therapy is to be delivered to a portion of the patient's body (e.g., such as a cardiac therapy delivered to the patient's heart 160) as indicated by block 510. The determination to deliver a therapy is generally based upon detection algorithms based on physiologic data acquired by the implantable medical device 110. Based upon such physiologic data received by data acquisition controller 440, the processor 410 determines whether a cardiac therapy is required. When a determination is made to deliver a therapy, the system 200 then determines the amount of energy to be delivered for the cardiac therapy (block 520) and the delivery sequence relative to the paced or intrinsic cardiac activity of the patient. In one embodiment, the processor 410 is preprogrammed to a predetermined intensity of the delivered therapy (e.g., from about one joule to about 30 joules for defibrillation therapy delivered by an implantable medical device). Thus, the processor 410 can be programmed and may be later modified using the access device 240 to control therapy delivery. In addition, the processor 410 may provide different: amounts of energy, types of waveforms and sequences of therapy delivery for a single type of arrthymia in order to effectively terminate or cardiovert, as applicable, the arrthymia, as is known in the art.

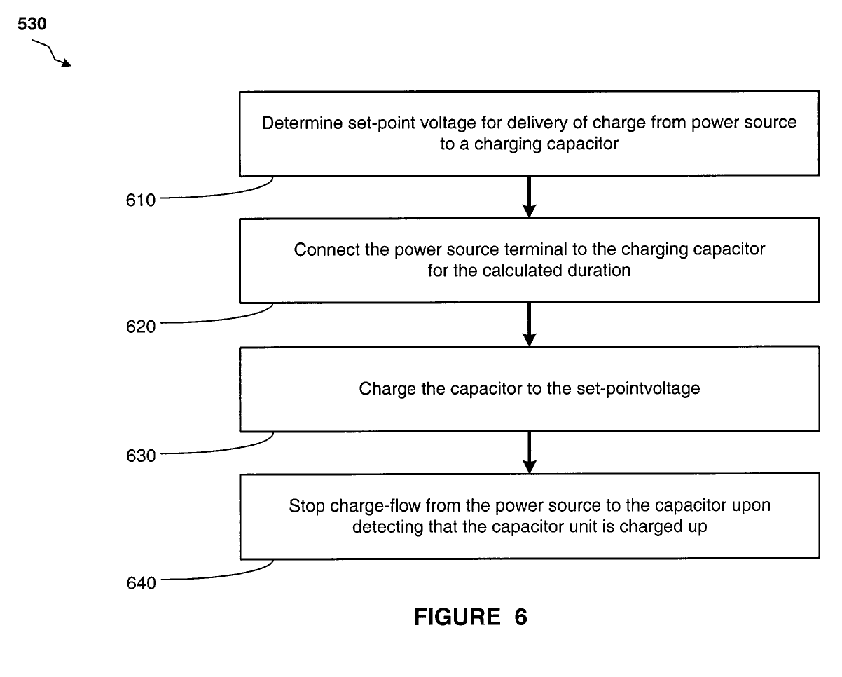

With continuing reference to FIG. 5, when a cardiac defibrillation therapy is to be delivered, transformer circuitry (not specifically depicted) draws electrical current from a high-rate battery cell to charge the capacitors for eventual discharge (block 530). A more detailed illustration and description of the step of charging up the capacitor unit 315 for eventual discharge (as indicated in block 530 in FIG. 5) is described in FIG. 6 and in the accompanying description below. The system 200 then prompts the control logic 420 to cause the power control unit 480 to rapidly charge the capacitor unit 315. The capacitor unit 315 is then charged to a predetermined voltage level (i.e., the set-point voltage). Upon completion of this initial charging of the capacitor unit 315, the system 200 performs a charge maintenance process to essentially maintain the full charge in the capacitor unit 315 (block 540).

In a related embodiment of the present invention, the medical device 110 comprises an external automatic defibrillator (AED). Operational AED circuitry monitors the voltage level of a capacitor unit during charging to a set-point voltage. After the voltage level of the capacitor unit completes the initial full-charging process a charge maintenance circuit begins to provide an electrical current to the capacitor unit as described above. The charge maintenance current works to maintain the voltage level of the capacitor unit fully charged. Preferably, this charge maintenance current continues to be applied so that voltage level of the capacitor unit remains fully charged to the predetermined threshold. In one form of this embodiment, the charge maintenance current may be interrupted or terminated immediately prior to delivery of the charge to the cardiac tissue. Of course, this form of the invention may also apply to implantable medical devices incorporating the present invention. In an alternative, albeit presently not preferred embodiment, the charge maintenance process may be invoked during, as well as afterwards, the initial charging of the capacitor 315.

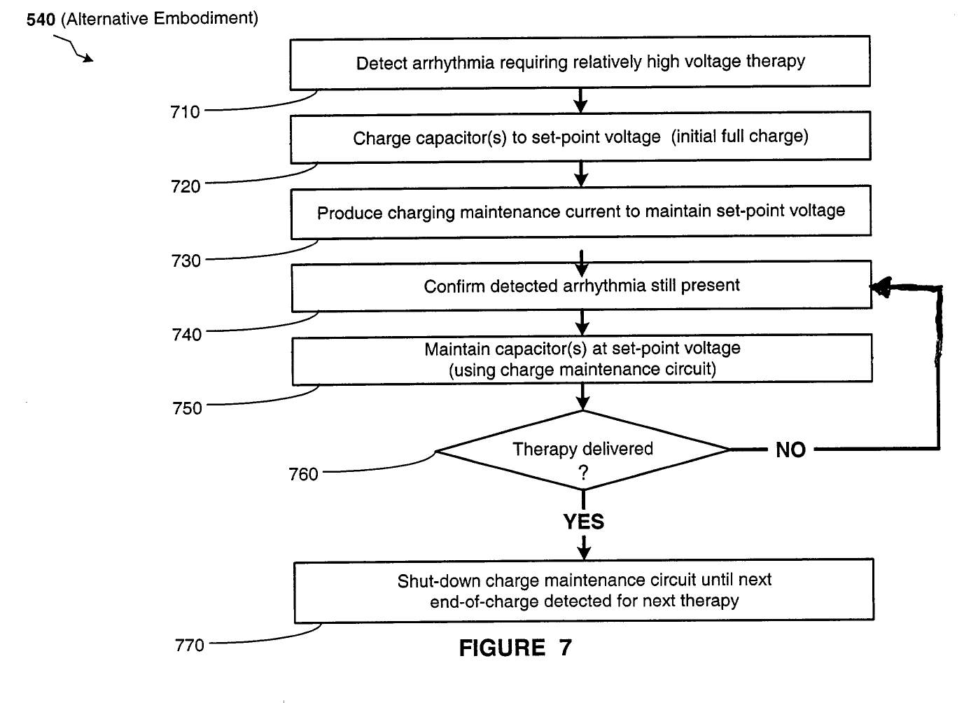

A more detailed description and illustration of an alternative method of performing the charge maintenance process indicated in block 540 of FIG. 5 is provided in FIG. 7, and in the accompanying description below. Upon performing the charge maintenance process the high voltage defibrillation therapy is delivered by the system 200 (block 550). As a result of performing the charge maintenance process, the predicted and delivered energy are the same. Once the therapy is delivered by the system 200, the system 200 continues to monitor physiologic data for a subsequent therapy (block 560) and/or begins to provide post-defibrillation pacing therapy to the patient, as applicable.

Turning now to FIG. 6, a flowchart depiction of the method of performing the charge of a capacitor unit 315 as indicated in block 530 of FIG. 5 is illustrated. The processor 410 in the implantable medical device 110 determines an appropriate set-point for delivery of the anticipated therapy delivery (block 610). The processor 410 then couples the power source 320 to the capacitor 315. In one embodiment, the power control unit 480 controls the connection between the power source 320 (as well as the timing and frequency of the electrical current delivered to the step-up transformer) and the capacitor unit 315. The power control unit 480 prompts the power source 320 to initiate current flow from the power source 320 to the capacitor unit 315 and continues said flow until the capacitor reaches the set-point voltage.

As described above, preferably upon completion of the primary charge of the capacitor unit 315 the charge maintenance process begins. Since conserving electrical power is a prime design consideration for chronic implantable medical device applications, the primary and maintenance (or secondary) charge processes, are preferably timed, monitored and/or controlled by the system 200 to maintain maximum efficiency. The completion of the steps indicated in FIG. 6 substantially completes the step of charging up capacitor unit 315 for eventual discharge, as indicated in block 530 of FIG. 5.

Turning now to FIG. 7, a flowchart depiction of an alternative method of performing the charge maintenance process (indicated in block 540 of FIG. 5) is illustrated. The delivery of therapy (e.g., cardiac pacing and/or anti-arrthymia therapy) is often timed by the implantable medical device 110 to safely coincide with certain physiological events (e.g., refractory period of the ventricles). Due to this timing, there is often a time lapse between the moment the capacitor unit 315 is charged up and the carefully timed delivery of therapy (i.e., especially the rapid discharge of the energy in the capacitor unit 315 during defibrillation therapy delivery). The implantable medical device 110 monitors the cardiac cycle following detection of an arrthymia (block 710) so that at the approximate moment following the initial charge process for the capacitor unit 315 (block 720) the charging maintenance current is applied to maintain the desired set-point voltage (block 730). Then, the presence of the arrthymia is reconfirmed (block 740). Following such confirmation, the charge maintenance circuit may optionally continue (block 750), for the anticipated brief amount of time prior to delivery of appropriately time therapy is delivered (block 760). As noted previously, the interval of time between completion of the charge (and reconfirmation of the arrthymia) and the ultimate delivery of the therapy varies. The implantable medical device 110 detects whether the set point voltage has been reached (block 740). In one embodiment, the detection of the set point voltage is performed by the charge maintenance unit 460. In response to detecting that a set point voltage has been reached, the implantable medical device 110 produces a charge maintenance current to maintain the set point voltage (block 750). The production of the charging current generally maintains the approximate desired charged-up voltage in the capacitor unit 315.

When the system 200 determines that the voltage level of the capacitor unit 315 equal to the set point voltage, the maintenance current is cut-off or reduced, as described in detail elsewhere in this disclosure. When the system 200 determines that the voltage level of the capacitor unit 315 below the set point voltage, the charging current is restarted until the voltage level of the capacitor unit 315 is equal to the set point voltage. The voltage level of the capacitor unit 315 is generally maintained for a predetermined period of time.

In one embodiment, the charge maintenance unit 460 then determines whether the cardiac therapy has been delivered (block 760). When there is an indication that the therapy has not been delivered, the charge maintenance unit 460 maintains the charging current in order to maintain the charge-up voltage in the capacitor unit 315, as indicated by the path from block 760 back to 750 in FIG. 7. When there is an indication that a cardiac therapy has been delivered (i.e., the capacitor unit 315 has been therapeutically discharged), the charge maintenance unit 460 preferably stops the charge maintenance current until the next charge-up of the capacitor unit 315, for a subsequent therapy, is detected (block 770). The steps described in FIG. 7 are used to maintain the integrity of the intensity and efficiency of the cardiac therapy delivered to the patient 112. The completion of the steps described in FIG. 7 substantially concludes the step of performing charge maintenance process described in block 540 of FIG. 5. The inventor recognizes that the principles described in the present disclosure can be implemented into a variety of electrical circuitry in addition to medical devices, but that these principles have particular utility within the medical device industry.

The particular embodiments disclosed above are illustrative only, as the invention may be modified and practiced in different but equivalent manners apparent to those skilled in the art having the benefit of the teachings herein. For example, the present invention may be embodied in software, firmware, hardware or combination thereof. As is also known in the art, the parameters (e.g., set point, charge rate, sequences for therapy delivery, etc.) may be preprogrammed and/or reprogrammable using programmable logic devices via various means of data telemetry and the like.

Furthermore, no limitations are intended with respect to the details of construction or design herein shown, other than as described in the claims below. It is therefore evident that the particular embodiments disclosed above may be altered or modified and all such variations are considered within the scope and spirit of the invention. Accordingly, the protection sought herein is set forth in the claims below.

What is claimed:

1. A method of maintaining a desired fully charged condition of a capacitor, comprising:
initially charging a capacitor to a predetermined set point voltage for delivery of a cardiac defibrillation therapy; and
applying a charge maintenance electrical current to the capacitor of magnitude sufficient to offset electrical leakage current from the capacitor and to counteract dipole alignment following said initial charging of the capacitor.

2. A method according to claim 1, wherein the magnitude of the charge maintenance electrical current dynamically varies in response to fluctuations in at least the electrical leakage current from the capacitor.

3. A method according to claim 2, wherein the charge maintenance electrical current is applied for less than about thirty seconds.

4. A method according to claim 3, wherein the capacitor comprises a stack of electrode plates formed of a valve metal material and a wet electrolyte in fluid communication with said plates.

5. A method according to claim 1, wherein the capacitor comprises a valve metal anode member, a high capacitance cathode and a wet or solid electrolyte.

6. A computer readable program storage medium encoded with instructions that, when executed by a computer, performs a method, comprising:
initially charging a capacitor to a predetermined set point voltage for delivery of a cardiac defibrillation therapy; and
applying a charge maintenance electrical current to the capacitor of magnitude sufficient to offset electrical leakage current from the capacitor and to counteract dipole alignment following said initial charging of the capacitor.

7. A method according to claim 6, wherein the magnitude of the charge maintenance electrical current dynamically varies in response to fluctuations in at least the electrical leakage current from the capacitor.

8. A method according to claim 7, wherein the charge maintenance electrical current is applied for less than about thirty seconds.

9. A precision capacitor charging apparatus for maintaining an initially charged capacitor at a set point voltage, comprising:
a processor;
a control logic operatively coupled to said processor, said control logic to generate at least one control signal in response to a command from said processor;
a capacitor operatively coupled to said control logic, said capacitor to acquire an initial full charge in response to said at least one control signal; and
a charge maintenance means operatively coupled to said capacitor, for providing a charge maintenance current to said capacitor unit after said initial full charge is acquired and continuing to provide said charge maintenance current until said capacitor is discharged in response to the at least one control signal from said control logic.

10. An apparatus according to claim 9, wherein said at least one control signal comprises: an initiate-initial-charge signal, a cease-initial-charge signal, a check leakage current signal, an initiate maintenance-charge signal, a discharge capacitor package signal.

11. An apparatus according to claim 10, wherein the control logic comprises at least one discharge waveform type.

12. An apparatus according to claim 11, wherein the at least one waveform type comprises at least a one of the following: a square wave, an exponentially decaying waveform, a biphasic waveform, a monophasic waveform, a chopped decaying waveform.
